(19) United States
(12) Patent Application Publication
(10) Pub. No.: US 2022/0008327 A1
(43) Pub. Date: Jan. 13, 2022

(54) Min Private Daily Care

(71) Applicant: Tova Palomino, Coral Springs, FL (US)

(72) Inventor: Tova Palomino, Coral Springs, FL (US)

(21) Appl. No.: 16/922,352

(22) Filed: Jul. 7, 2020

(51) Int. Cl. A61K 8/9789; A61F 13/40; A61Q 19/00; A61K 8/9794

(52) U.S. Cl. CPC A61K 8/9789; A61M 35/006; A61M 2210/167; A61K 8/9794; A61M 2210/1475; A61Q 19/007

(57) ABSTRACT

The present invention discloses an organic skin moisturizer for vagina and penis, which extends and improves the skin cells. The composition is dermatologically acceptable and is capable of moisturizing skin to prevent infections and support a healthy pH balance while enhancing the overall feel and appearance.

### BACKGROUND

### Copyright Notice

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention pertains generally relates to cosmetic preparations for the moisturization of human skin. More particularly, the present invention is particularly, but not exclusively, useful for a moisturizing oil suitable for use on the vagina and penis.

### DESCRIPTION OF THE RELATED ART

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof to anything stated or implied therein or inferred thereupon.

A lubricating fluid is normally present in the vagina and during sexual arousal an increased amount of the fluid is produced. The lack of sufficient vaginal lubrication causes vaginal tissue to become dry and irritated and may result in painful sexual intercourse and sometimes bleeding. There are a number of causes for this condition including decreased estrogen levels during menopause or after surgical removal of ovaries and after radiation therapy. Oral contraceptives and certain medications such as antihistaminics, antidepressants, blood pressure, and cardiac medicines can contribute to vaginal dryness. Additionally, psychological conditions including stress, fatigue and anxiety may impede production of the natural lubricant. A combination of hormonal and psychological factors may induce dryness temporarily after childbirth particularly if the mother is breastfeeding.

Common personal lubricants including K-Y Jelly and Vaseline are not ideal due to their consistency, poor lasting quality and lack of pleasant fragrance and acceptable taste. Those containing glycerin cause decreased sperm motility. Additionally, commercially available lubricants deal only with lubricating and do not contain ingredients that promote healing of vaginal tissue if irritation and inflammation are present.

Vaginal lubricating suppositories are sold commercially, however, they must be inserted into the mid-vagina to liquify adequately and then provide lubrication. There is a delay before the suppository melts when and where it is most needed.

Foam-type vaginal lubricants are also sold commercially. Some are packaged wider pressure in containers similar to shaving cream cans. Their use has poor acceptability because of the emission sound and they provide lubrication for only a relatively short time.

However, what has not been obvious so far is our finding that inclusion of such agents in lubricant compositions improves the viability, motility and fertilization potential of the sperm that come in contact with lubricants and moisturizers. A composition that improves viability, motility and/or mucus penetration of sperm after transfer to a female should greatly improve the chances of fertilization. Such novel compositions are also non-toxic to the oocyte and the fertilized embryo. The novel compositions of this invention are particularly useful as lubricants for use prior to or during coitus. An object of this invention is to provide systems, articles, and methods of manufacture and use of such novel compositions to facilitate fertilization.

In light of the above it is an object of the present invention to provide a safe and effective penis or vaginal moisturizer product in a novel delivery form to meet the above-mentioned needs of the population of women who suffer from vaginal dryness.

The present invention provides a new moisturizer form having many of the properties of an ideal moisturizer, None of the prior art methods have been found to be completely suitable to meet these needs and are cumbersome. The present invention provides such a method and the overall combination of these features is nowhere disclosed in the prior art cited above which appears to be representative of the general art in this area although it is not intended to be an all-inclusive listing of pertinent prior art patents.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, and abstract as a whole.

The present invention is a penis and vaginal moisturizer which does not impede sperm motility and may be used when infertility is present.

Another embodiment of the invention is a mixture of oils and essential oils which is to help moisturize and aid in common skin problems.

Another object of the present invention is to provide a cosmetic preparation such as moisturizer which produce products that have long lasting effects on the skin and results in longer effective treating times for any active ingredient which may be added.

In an alternative embodiment, the present invention effectively softens, moisturizes and provides a unique essential oils combination of needed nutrients to the skin.

Advantage of this invention. A vaginal or penis topical lotion or Crème according to the present invention soothes irritated inflamed vaginal tissue when present. The invention is aesthetically acceptable in application and use, is applied externally to either partner, is nonspermicidal and does not impede sperm motility.

It is principal object of the invention, by electively softening, moisturizing, and supplying appropriate vitamins to the skin of penis or vagina.

Another significant embodiment of the present invention provide an advantageously a single composition which moisturizes the skin and protect it at the same time. Thus, it is more convenient for a subject in need of such a composition and advantageously provide a great compliance, One embodiment of the present invention is a moisturizing composition comprising at least one moisturizer ingredient.

It is an object of the invention to provide a new skin moisturizer which may be easily and efficiently manufactured and marketed and which is of an effective and reliable construction. According to another aspect of the invention, it is provided a method far preventing or treating vaginal dryness, comprising the step of applying the genital composition.

The essential oils used herein according to present invention is selected from:

### Olea Europaea Fruit Oil (Olive)

### Melaleuca Alternifolia Oil (Tea Tree)

### Cananga Odorata Flower Oil (Ylang Ylang)

### Mentha Piperita Oil (Peppermint)

### Boswellia Carterii Oil (Frankincense)

### Anthemis Nobilis Oil (Roman Chamomile)

### Rosa Moschata Seed Oil (Rosehip)

### Thymus Vulgarius Oil (Thyme)

### Allium Sativum Oil (Garlic)

The benefits of the present invention are as follows:

Balances hormones

### Balances PH

Protects against bacteria and infections
Improves blood circulation
Evens skin tone
Prevents ingrown hairs

### Anti-viral

### Anti-fungal

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, and Claims.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. Furthermore, there is no intention to be bound by any expressed or implied theory presented in the preceding technical field, background, brief summary or the following detailed description. It is also to be understood that the specific devices and processes described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

Unless otherwise defined, all terms (including technical and scientific terms) used herein have the same meaning as commonly understood by one having an ordinary skill in the art to which the invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques are disclosed. Each of these has individual benefit and each can also be used in conjunction with one or more, or in some cases all, of the other disclosed techniques. Accordingly, for the sake of clarity, this description will refrain from repeating possible combination in an unnecessary fashion. Nevertheless, the specifications and claim/s should be read with the understanding that such combinations are entirely within the scope of the invention and the claim/s.

The method of the present invention is that it relates to a moisturizer oil which is to be applied to the vagina and penis. The formula is a mixture of oils and essential oils which is to help moisturize and aid in common skin concerns. The particular formula consists of the following:

### Olea Europaea Fruit Oil (Olive)

### Melaleuca Alternifolia Oil (Tea Tree)

### Cananga Odorata Flower Oil (Ylang Ylang)

### Mentha Piperita Oil (Peppermint)

### Boswellia Carterii Oil (Frankincense)

### Anthemis Nobilis Oil (Roman Chamomile)

### Rosa Moschata Seed Oil (Rosehip)

### Thymus Vulgarius Oil (Thyme)

### Allium Sativum Oil (Garlic)

The benefits of the present invention are as follows:

Balances hormones

### Balances PH

Protects against bacteria and infections
Improves blood circulation
Evens skin tone
Prevents ingrown hairs

### Anti-viral

### Anti-fungal

Another embodiment of the present invention relates to simple application where the moisturizing skin cream can be applied daily. To apply, a generous amount is first placed on the fingertips or an appropriate delivery device such as a sponge or cloth. Next, the cream is dabbed onto the skin at the area to be treated. Finally, the cream is rubbed into the skin using the fingertips until the cream becomes transparent. Use of the skin cream immediately after washing and towel drying the skin is recommended for maximum moisturizing effect.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Moreover, a latitude of modification, change, and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

1) The invention is a formula of the facial moisturizer with essential oils comprising:
a. Olea Europaea Fruit Oil (Olive);
b. Melaleuca Alternifolia Oil (Tea Tree);
c. Cananga Odorata Flower Oil (Ylang Ylang);
d. Mentha Piperita Oil (Peppermint);
e. Boswellia Carterii Oil (Frankincense);
f. Anthemis Nobilis Oil (Roman Chamomile);
g Rosa Moschata Seed Oil (Rosehip);
h. Thymus Vulgarius Oil (Thyme);
i. Allium Sativum Oil (Garlic).

2) wherein the invention is Nourishing face moisturizing oil, Revitalizing hand and body moisturizer providing the following benefits:
a. Balances hormones;
b. Balances PH;
c. Protects against bacteria and infections;
d. Improves blood circulation;
e. Evens skin tone;
f. Prevents ingrown hairs;
g. Anti-viral;
h. Anti-fungal.

3) The invention can be used multiple times a day and preferably for use in morning and night.
